(19) United States
(12) Patent Application Publication
(10) Pub. No.: US 2024/0173363 A1
(43) Pub. Date: May 30, 2024

(54) MICROBIOME COMPOSITION OF HALOPHILIC BACILLUS VELEZENSIS KMU01 STRAIN FERMENTED CULTURE SUPERNATANT WITH ANTI-OBESITY EFFECT

(71) Applicant: KOOKMINBIO, CORP., Jeonju-si (KR)

(72) Inventor: Moon-Hee SUNG, Seoul (KR)

(21) Appl. No.: 18/489,830

(22) Filed: Oct. 18, 2023

(30) Foreign Application Priority Data
Oct. 18, 2022 (KR) 10-2022-0134157
Oct. 12, 2023 (KR) 10-2023-0135572

(51) Int. Cl. A61K 35/742; A61K 31/198; A61P 3/04

(52) U.S. Cl. CPC A61K 35/742; A61K 31/198; A61P 3/04

(57) ABSTRACT

The present disclosure relates to a microbiome composition of a halophilic Bacillus polyfermenticus KMU01 strain fermented culture supernatant with an anti-obesity effect. It was found that a fermented culture supernatant of the strain deposited in deposit number KCTC11751BP suppresses generation and accumulation of fat and reduces a content of blood cholesterol, such that the fermented culture supernatant may be useful as a composition for preventing, treating, or ameliorating obesity; or a composition for reducing body fat (visceral fat) or blood cholesterol.

### CROSS-REFERENCE TO RELATED APPLICATION

This application claims the benefit of Korean Patent Application No. 10-2023-0135572 filed on Oct. 12, 2021, in the Korean Intellectual Property Office, the entire disclosure of which is incorporated herein by reference for all purposes.

### BACKGROUND

### 1. Field of the Invention

The present disclosure relates to a microbiome composition of a halophilic Bacillus velezensis KMU01 strain fermented culture supernatant with an anti-obesity effect.

### 2. Description of the Related Art

Obesity is one of the most representative diseases with aspects that are rapidly changing to a type of developed countries, as sanitary environment is improved with enhancement in living standards in recent years while the average life expectancy has been extended due to a westernized diet. Therefore, adult diseases have emerged as the biggest medical issues today, and obesity, which is the main cause of these adult diseases, is also rapidly increasing.

Obesity is a disease caused by an imbalance between food intake and energy consumption, referring to a condition with an excessive increase in adipose tissues. The continuation of obesity leads to various diseases such as hypertension, elevated blood cholesterol, kidney disease, stroke, arteriosclerosis, fatty liver, arthritis, cancer, sleep apnea, and diabetes, of which accumulation of visceral fat in the abdominal fat causes insulin resistance or increased lipogenesis in the liver to cause metabolism abnormalities for sugar and lipid, hypertension, and coronary artery disease, such that the importance in the treatment of obesity is increasing.

Therapeutic agents for obesity are generally divided into three categories that are appetite suppressants, promoters of body energy metabolism, and inhibitors of digestion and absorption. A representative therapeutic agent for obesity involving a pharmacological mechanism to suppress appetite is Reductil™ (Abbott, USA), that involving a pharmacological mechanism to promote body energy is Exorise™ (ACO Pharma, France), and that involving a pharmacological mechanism to inhibit fat digestion and absorption is Xenical™ (Roche Pharma, Switzerland).

Recently, thanks to the development of next-generation sequencing (NGS) technology, many studies have been conducted on microorganisms present in the human body, and studies to prove relation between obesity and the gut microbiome are also being actively conducted as well. The gut microbiome is closely related to the host's diet, and it has been reported that obesity changes gut microbiota and functions, leading to dysbiosis in gut microbes. In fact, transplantation of gut microbes from obese mice into normal or sterile mice resulted in weight gain and metabolic disorders, which have been reported to have relation of gut microbes with dietary energy utilization and metabolic regulation of fat and fatty acid in hepatic tissues. Therefore, regulation of the gut microbiota may be a non-toxic, safe, and potential treatment for metabolic disorders in terms of amelioration of obesity.

While probiotics have instability and issues in reaching the intestines despite effects of treating and preventing immune diseases by preventing the access of pathogenic microorganisms by regulating the gut flora, cell-free supernatants (CFSs) are becoming popular as a new alternative material that may overcome safety and functionality issues concerning side effects due to excessive intake. Recently, cultures of Bifidobacterium bifidum DS0908 and Bifidobacterium bifidum DS0950 have been shown to reduce obesity by promoting thermogenesis in obese mice, with reports that short-chain fatty acid (SCFA) prevents obesity caused by a high-fat diet by regulating intestinal hormones. As such, various studies on the physiological activity of strain cultures are underway.

### PRIOR ART DOCUMENT

### [Patent Document]

(Patent Document 1) 1. Korean Patent No. 10-2176920 (issued on Nov. 4, 2020)

### Non-Patent Documents

(Non-Patent Document 1) 1. Functional Annotation Genome Unravels Potential Probiotic Bacillus velezensis Strain KMU01 from Traditional Korean Fermented Kimchi (DOI: https://doi.org/10.3390/foods10030563, published on May 9, 2021)
(Non-Patent Document 2) 2. Genome Sequence of the Probiotic Strain Bacillus velezensis Variant polyfermenticus GF423 (DOI: 10.1128/MRA.01000-18, published on Sep. 13, 2018)

### SUMMARY

### Problem to be Solved by the Invention

An object of the present disclosure is to provide a method of preventing, treating or ameliorating obesity.

Another object of the present disclosure is to provide a method of reducing body fat or blood cholesterol.

### Means for Solving the Problem

To achieve the above objects, the present disclosure provides a method of preventing or treating obesity, comprising: administering a pharmaceutical composition comprising a fermented culture supernatant of a Bacillus velezensis strain, a concentrate thereof, a dried product thereof, a fermentation metabolite thereof, or a mixture thereof as an active ingredient to a subject.

In addition, the present disclosure provides a method of preventing or ameliorating obesity, comprising: administering a health functional food composition comprising a fermented culture supernatant of a Bacillus velezensis strain, a concentrate thereof, a dried product thereof, a fermentation metabolite thereof, or a mixture thereof as an active ingredient to a subject.

In addition, the present disclosure provides a method of preventing or ameliorating obesity, comprising: administering a food composition comprising a fermented culture supernatant of a Bacillus velezensis strain, a concentrate thereof, a dried product thereof, a fermentation metabolite thereof, or a mixture thereof as an active ingredient to a subject.

In addition, the present disclosure provides a method of reducing body fat or blood cholesterol, comprising: administering a health functional food composition comprising a fermented culture supernatant of a Bacillus velezensis strain, a concentrate thereof, a dried product thereof, a fermentation metabolite thereof, or a mixture thereof as an active ingredient to a subject.

### Effects of the Invention

According to the present disclosure, it was found that a fermented culture supernatant of a Bacillus velezensis KMU01 strain deposited in deposit number KCTC11751BP suppresses generation and accumulation of fat and reduces a content of blood cholesterol, such that the fermented culture supernatant may be useful as a method for preventing, treating, or ameliorating obesity; or a method for reducing body fat (visceral fat) or blood cholesterol.

### BRIEF DESCRIPTION OF THE DRAWINGS

FIG. 1 is a schematic diagram showing a process of adipogenesis using a 3T3-L1 preadipocyte.

FIG. 2 shows an image of an adipose tissue extraction process in a mouse animal model.

FIG. 3 shows a schematic diagram illustrating a gut microbe analysis process in a mouse animal model.

FIG. 4 shows a result of analyzing cytotoxicity of a strain culture (hereinafter referred to as a sample) of Bacillus velezensis KMU01 (hereinafter referred to as KMU01) in adipocytes.

FIG. 5 shows results of analyzing an effect of a sample on accumulation of fat and triglycerides (hereinafter referred to as TG).

FIG. 6 shows results of analyzing an effect of a sample on expression of adipogenesis-related genes and lipogenesis-related enzymes (hereinafter referred to as fatty acid synthase (FAS)).

FIG. 7 shows results of analyzing an effect of a sample on a weight and dietary efficiency in animal models.

FIG. 8 shows results of analyzing an effect of a sample on a body composition in animal models.

FIG. 9 shows results of analyzing an effect of a sample on organs and adipose tissues in animal models. iWAT; inguinal white adipose tissue, mWAT; mesenteric white adipose, rWAT; retroperitoneal white adipose tissue and eWAT; epididymal white adipose tissue.

FIG. 10 shows results of analyzing an effect of a sample on adipose tissues in animal models.

FIG. 11 shows a result of analyzing an effect of a sample on hepatic TG in animal models.

FIG. 12 shows results of analyzing an effect of a sample on expression of proteins related to hepatic adipogenesis and lipogenesis in animal models.

FIG. 13 shows results of analyzing an effect of a sample on gut microbes in animal models.

In all diagrams, there is a statistical difference at a 95% confidence level between treatment groups indicated with different alphabets (ex. a, b, etc.). For example, if there is group 1 marked a, group 2 marked b, and group 3 marked ab, there is a statistical difference at a level of 95% between groups 1 and 3, with no statistical difference between groups 1 and 2; and between groups 2 and 3.

### DETAILED DESCRIPTION

Hereinafter, the present disclosure will be described in detail.

The present disclosure provides a method of preventing or treating obesity, comprising: administering a pharmaceutical composition comprising a fermented culture supernatant of a Bacillus velezensis strain, a concentrate thereof, a dried product thereof, a fermentation metabolite thereof, or a mixture thereof as an active ingredient to a subject.

The pharmaceutical composition may reduce visceral fat production.

In addition, the pharmaceutical composition may regulate, but is not limited to, one or more gut microbes selected from the group consisting of Acetatifactor muris, Mucispirillum schaedleri, and Eubacterium plexicaudatum.

In addition, the pharmaceutical composition may reduce visceral fat production by decreasing relative abundance of an Acetatifactor muris or Mucispirillum schaedleri strain in the intestine and increasing relative abundance of an Eubacterium plexicaudatum strain in the intestine.

The strain may be a Bacillus velezensis KMU01 strain deposited in deposit number KCTC11751BP.

The strain name at the time of deposition of the Bacillus velezensis KMU01 strain is Bacillus polyfermenticus KMU01. Specifically, the KMU01 strain was isolated as Bacillus amyloliquefaciens in 2010 and reclassified as Bacillus polyfermenticus in 2018 based on the 16S rRNA gene sequence. Subsequently, an experiment conducted to identify the exact species of the KMU01 strain revealed that the gene sequence of the KMU01 strain showed a similarity of 97.7% with Bacillus velezensis, and now the KMU01 strain was determined to be Bacillus velezensis (Functional Annotation Genome Unravels Potential Probiotic Bacillus velezensis Strain KMU01 from Traditional Korean Fermented Kimchi, DOI: https://doi.org/10.3390/foods10030563, published on May 9, 2021).

Recently, the Bacillus polyfermenticus KMU01 strain was renamed Bacillus velezensis (Genome Sequence of the Probiotic Strain Bacillus velezensis Variant polyfermenticus GF423, DOI: 10.1128/MRA.01000-18, published on Sep. 13, 2018).

In addition, the pharmaceutical composition may further include dead bacteria or spores of Bacillus velezensis.

The fermentation metabolite may be short chain fatty acids (SCFAs), organic acids, or amino acids.

The short chain fatty acid may be butyric acid or propionic acid, and the amino acid may be phenylalanine, which is an aromatic amino acid, or valine, which is a branched amino acid, but are not limited thereto.

In addition, the pharmaceutical composition may regulate adiponectin secretion.

In addition, the pharmaceutical composition may inhibit one or more expressions selected from the group consisting of, but is not limited to, peroxisome proliferator-activated receptor γ, CCAAT/enhancer binding protein α (C/EBPα), sterol regulatory element-binding protein-1c (SREBP-1c), fatty acid synthase (FAS), acetyl-CoA carboxylase (ACC), stearoyl-CoA desaturase-1 (SCD-1), and diacylglycerol acyltransferase (DGAT).

The obesity may be one or more selected from the group consisting of, but is not limited to, visceral obesity, abdominal obesity, systemic obesity, and partial obesity.

The pharmaceutical composition the present disclosure may be prepared in a unit dose form or prepared by infusion in a multi-dose container through preparation using pharmaceutically acceptable carriers according to a method that may be easily carried out by a person skilled in the art to which the present disclosure pertains.

The pharmaceutically acceptable carriers are those commonly used in preparation, including lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methyl cellulose, methyl hydroxybenzoate, propyl hydroxybenzoate, talc, magnesium stearate, and mineral oil, but are not limited to. The pharmaceutical composition of the present disclosure may further include lubricants, wetting agents, sweetening agents, flavoring agents, emulsifying agents, suspending agents, and preservatives, in addition to the above components.

In the present disclosure, the content of additives included in the pharmaceutical composition is not particularly limited and may be appropriately adjusted within the content range used for conventional preparation.

The pharmaceutical composition may be formulated in the form of one or more external skin preparations selected from the group consisting of, but is not limited to, injectable formulations such as aqueous solutions, suspensions, and emulsions, pills, capsules, granules, tablets, creams, gels, patches, sprays, ointments, emplastrum agents, lotions, liniments, pastas, and cataplasmas.

The pharmaceutical composition of the present disclosure may include pharmaceutically acceptable carriers and diluents that are additional for the formulation. The pharmaceutically acceptable carrier and diluent include excipients such as starch, sugar, and mannitol, fillers and extenders such as calcium phosphate, cellulose derivatives such as carboxymethylcellulose and hydroxypropyl cellulose, binders such as gelatin, alginate, and polyvinylpyrrolidone, lubricants such as talc, calcium stearate, hydrogenated castor oil, and polyethylene glycol, disintegrants such as povidone and crospovidone, and surfactants such as polysorbates, cetyl alcohol, and glycerol, but are not limited thereto. The pharmaceutically acceptable carrier and diluent may be biologically and physiologically compatible with subjects. Examples of the diluent may include saline, aqueous buffers, solvents, and/or dispersion media, but are not limited thereto.

The pharmaceutical composition of the present disclosure may be administered orally or parenterally (e.g., intravenously, subcutaneously, intraperitoneally, or topically) depending on a desired method. For oral administration, the pharmaceutical composition may be formulated as tablets, troches, lozenges, aqueous suspensions, oily suspensions, powder preparation, granules, emulsions, hard capsules, soft capsules, syrups, and elixirs. For parenteral administration, the pharmaceutical composition may be formulated as injections, suppository agents, powder for respiratory inhalation, aerosols for sprays, ointments, powder for application, oil, and creams.

The dosage range of the pharmaceutical composition of the present disclosure may vary depending on the patient's condition, body weight, age, sex, health status, dietary constitution specificity, the nature of preparations, the severity of diseases, administration duration of the composition, administration methods, administration periods or intervals, excretion rate, and drug forms, and be appropriately selected by those skilled in the art. For example, the dosage may be in the range of about 0.1 to 10,000 mg/kg but is not limited thereto, while it may be administrated in divided doses from one to several times a day.

The pharmaceutical composition may be administered orally or parenterally (e.g., intravenously, subcutaneously, intraperitoneally, or topically) depending on a desired method. A pharmaceutically effective amount and effective dosage of the pharmaceutical composition of the present disclosure may vary depending on preparation methods, administration methods, administration duration, and/or administration routes of the pharmaceutical composition, and those skilled in the art may easily determine and prescribe the dosage effective for desired treatment. Administration of the pharmaceutical composition of the present disclosure may be conducted once a day or several times in divided doses.

In addition, the present disclosure provides a method of preventing or ameliorating obesity, comprising: administering a health functional food composition comprising a fermented culture supernatant of a Bacillus velezensis strain, a concentrate thereof, a dried product thereof, a fermentation metabolite thereof, or a mixture thereof as an active ingredient to a subject.

In addition, the present disclosure provides a method of reducing body fat or blood cholesterol, comprising: administering a health functional food composition comprising a fermented culture supernatant of a Bacillus velezensis strain, a concentrate thereof, a dried product thereof, a fermentation metabolite thereof, or a mixture thereof as an active ingredient to a subject.

The present disclosure may be generally used as a commonly used food product.

The food composition of the present disclosure may be used as a health functional food. The term “health functional food” as used herein refers to food manufactured and processed with raw materials or ingredients having useful functionality for the human body in accordance with the Health Functional Food Act, and the term “functionality” as used herein refers to the intake to derive effectiveness in health care such as physiological actions or regulation of nutrients for the structure and function of the human body.

The health functional food composition may include common food additives, and the suitability as the “food additive” is determined by the standards and criteria related to corresponding items according to the general rules and general test methods of Korean Food Additives Codex approved by the Ministry of Food and Drug Safety, unless otherwise stipulated.

The items listed in the “Korean Food Additives Codex” may include, for example, chemically synthesized compounds such as ketones, glycine, potassium citrate, nicotinic acid, and cinnamic acid, natural additives such as persimmon color, licorice extracts, crystallized cellulose, kaoliang color, and guar gum, and mixed preparations such as sodium L-glutamate preparations, noodle-added alkali agents, preservative agents, and tar color agents.

The food composition of the present disclosure may be manufactured and processed in the form of tablets, capsules, powder, granules, liquids, and pills. For example, hard capsule preparations among health functional foods in the form of capsules may be prepared by mixing and filling the composition according to the present disclosure in conventional hard capsules along with additives such as excipients, and the soft capsule preparations may be manufactured by mixing the composition according to the present disclosure with the additives such as excipients and then filling the same in capsule bases such as gelatin. The soft capsule preparations may include, if necessary, plasticizers such as glycerin or sorbitol, colorants, and preservatives.

The definition of terms for the excipient, binder, disintegrant, lubricant, flavor enhancer, and flavoring agent is described in documents known in the art and includes those having the same or similar functions. The type of food is not particularly limited and includes all health functional foods in the ordinary sense.

The term “prevention” as used herein refers to any action of suppressing or delaying obesity by administering the composition according to the present disclosure.

The term “treatment” as used herein refers to any action that improves or favorably changes the symptoms of obesity by administering the composition according to the present disclosure.

The term “improvement” as used herein refers to any action that improves the bad state of obesity by making an individual intake the composition of the present disclosure or administering the same.

In addition, the present disclosure provides a method of preventing or ameliorating obesity, comprising: administering a food composition comprising a fermented culture supernatant of a Bacillus velezensis strain, a concentrate thereof, a dried product thereof, a fermentation metabolite thereof, or a mixture thereof as an active ingredient to a subject.

Hereinafter, example embodiments will be described in detail to help the understanding of the present disclosure. However, the following example embodiments are merely illustrative of the content of the present disclosure, and the scope of the present disclosure is not limited to the following example embodiments. The example embodiments of the present disclosure are provided to more completely explain the present disclosure to those skilled in the art.

### Experimental Example 1

### Preparation of Samples

To prepare the sample, KMU01 (Bacillus velezensis KMU01) strain deposited in stock deposit number KCTC11751BP stored in a Working cell bank at −70° C. was activated and subjected to the primary strain culture in a test tube and flask, inoculation was performed by 2% (v/v) in a working volume of 20 L in a 50 L fermentation tank, and the secondary strain culture was carried out for 6 hours. This culture was performed for 12 hours by inoculating by 2% (v/v) in a working volume of 350 L in a 500 L fermentation tank, and glucose was additionally fed once at 6 hours of culture. After the culture is over, the cell slurry was removed by performing primary centrifugation at 7200 rpm and 2 L/min in a disk centrifuge, and the supernatant was subjected to secondary centrifugation twice at 15000 rpm and 1.5 L/min using a tubular centrifuge to remove the cell cake, followed by recovery of the supernatant. The recovered supernatant was filtered through a 0.2 μm antibacterial filter to obtain a sample from which bacteria were finally removed.

### Example 2

### In Vitro Experiment

### 2-1. Cell Culture and Differentiation

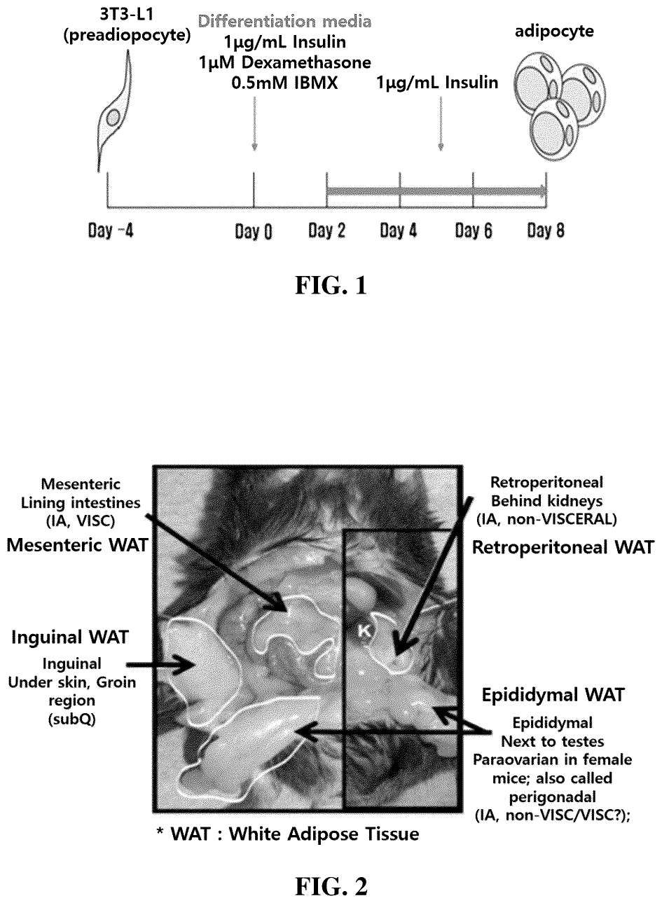

3T3-L1 (ATCC, Manassas, VA, USA) fibroblasts, which are preadipocytes, were cultured at 37° C. under 5% CO₂ conditions in Dulbecco's modified Eagle's medium (DMEM) including 10% (v/v) bovine calf serum and 100 μg/mL penicillin-streptomycin. Subsequently, as shown in FIG. 1, when the cells became 100% confluent, differentiation into adipocytes was induced with DMEM including 10% fetal bovine serum (FBS), 1 μM dexamethasone, 0.5 mM isobutyl methylxanthine (IBMX), 1 g/mL insulin, and 100 μg/mL penicillin-streptomycin. After 2 days of differentiation, medium replacement was performed with 10% FBS medium including 1 μg/mL insulin and the sample (medium was replaced every other day), and the effect on adipocyte proliferation and differentiation was analyzed at day 8.

### 2-2. Cytotoxicity Analysis

In order to identify cytotoxicity of the sample in adipocytes, MTT assay was performed. Adipocytes (3T3-L1) were treated with the samples by concentration (75, 150, and 300 μg/mL), and MTT assay was performed to measure cell viability.

### 2-3. Analysis of Fat Accumulation and TG Content

To determine the effect of the sample on fat accumulation, adipocytes (3T3-L1) were treated with the sample by concentration (75, 150, and 300 μg/mL), and adipocytes were stained by Oil-Red O (ORO) staining to measure a fat accumulation rate of mature adipocytes.

In addition, in order to identify the effect of the sample on TG, a content of TG accumulated in the cell was measured using a TG quantification kit (Abcam, Cambridge, MA, USA), the protein content was quantified through bicinchoninic acid (BCA) analysis, and then the TG content in cells was expressed by correcting by the protein concentration.

### 2-4. Analysis of Adipogenesis-Related Genes and FAS Expression

In order to identify the effect of the sample on adipogenesis-related genes and FAS expression, RNA was extracted using Nucleozol (Macherey-Nagel, Duren, Germany) reagent, and cDNA was synthesized using a reverse transcription kit (Applied Biosystem, Foster city, CA, USA). Subsequently, expression of adipogenesis-related genes (PPARγ, C/EBPα, and SREB-1c) and FAS was analyzed using the StepOnePlus Real-Time PCR (Quantitative real-time PCR; qPCR) system (Applied Biosystem), and a gene expression level was corrected using the glycealdehyde-3-phosphate dehydrogenase (GAPDH) gene.

### 2-5. Statistical Analysis

All quantitative analyzes were repeated three times. For Statistical analysis, SPSS (SPSS Inc., USA) software was used, and when significant differences were found in one-way ANOVA (P<0.05), Duncan's multiple comparison method was performed to test significant differences between treatment groups.

### Example 3

### In Vivo Experiment

### 3-1. Preparation of Animal Models

5-week-old male C57BL/6J mice were purchased from RAONBIO Inc., Republic of Korea. When brought in, the animal was inspected and weighed. General symptoms were observed once a day during the acclimatization period, and the health status of the animals was evaluated by checking the general symptoms and weight changes after measuring the weight at the end of the acclimatization period. In order to ensure evenness in the average weight of each experimental group, about 10 animals per group were separated into a total of 6 groups, and 5 animals were bred per cage. A five-colored oil-based pen was used to mark the animal's tail, and an individual identification card was attached to the breeding box. Animal models were bred for 2 weeks under conditions with temperature at 21 to 23° C., 40 to 60% relative humidity, and light and dark cycle of 12 hours/day (8 a.m. to 8 p.m.), with feed and drinking water supplied. The feed used therefor was laboratory animal feed (6% fat feed and 45% fat feed) (ENVIGO, RESEARCH DIETS INC.). The animal experiment was approved by the Institutional Animal Care and Use Committee of Kookmin University (KMU-2022-01) and carried out in accordance with the Standard Operating Procedure of Kookmin University.

### 3-2. Analysis of Weight and Dietary Efficiency

In order to identify the effect of the sample on the weight and dietary efficiency in the animal model, the animal model (7 weeks of age) prepared in above Experimental Example 3-1 was set into 4 groups as follows, the weight and feed intake were measured at intervals of one week for 13 weeks, and dietary efficiency was calculated using Equation 1 below. Xenical was used as a positive control. Samples and Xenical were administered orally in the stomach once daily for 13 weeks from the date of initiation of administration using a disposable syringe with a sonde for oral administration attached.
1) Normal diet group (NOR): 6% fat feed group
2) High-fat diet group (HFD): 45% fat feed group
3) Sample administration group (B. vele): a group of a high-fat diet group (HFD) orally administered with a sample (114 mg/kg/day)
4) Positive control group (Xen): a group of a high-fat diet group (HFD) administered with Xenical (50 mg/kg/day)

Food efficiency ratio=Weight gain (g/week)/Food intake (g/week)×100 [EQUATION 1]

### 3-3. Analysis of Body Composition

To identify the effect of the sample on the body composition of the animal model, a dual energy X-ray absorptiometry (InAlyzer; Medikors Inc., Seongnam, Korea) was used to measure the changes in the body composition in animal models at 13 weeks before sacrifice. During the analysis of the body composition, ketamine (100 mg/kg BW) and xylazine (10 mg/kg BW) were injected into animal models for anesthesia, followed by measurement. When ketamine having an anesthetic effect is used alone, side effects due to muscle contraction may occur during the recovery process from anesthesia, such that xylazine, a muscle relaxant, was used along.

### 3-4. Analysis of Weights of Organs and Adipose Tissues

In order to identify the effect of the sample on the organs and adipose tissues of the animal model, the animal model was sacrificed by fasting for 18 hours and then dissected to extract the heart, liver, kidneys, and spleen, and the organs were weighed. As shown in FIG. 2, adipose tissues were separated into subcutaneous fat, mesenteric fat, retroperitoneal fat, and epididymal fat, followed by weight measurement.

### 3-5. Biochemical Analysis of Blood

In order to identify the effect of the sample on blood glucose, aspartate aminotransferase (GOT; hereinafter referred to as AST), alanine aminotransferase (GPT; hereinafter referred to as ALT), blood urea nitrogen (hereinafter referred to as BUN) and cholesterol in the animal model, blood was collected from the heart after sacrifice of the animal model and immediately centrifuged (2000×g, 10 minutes) to separate the plasma, and the plasma was stored in a deep freezer at −80° C. until analysis. Blood glucose, AST, ALT, and BUN were measured using a chemical analyzer (Fuji DRI-CHEM 3500i, Fuji Photo Film, Ltd., Tokyo, Japan), total cholesterol and high density lipoprotein (HDL)-cholesterol (hereinafter referred to as HDL-C) were measured using a LabAssay™ Cholesterol kit (Wako, Osaka, Japan), and a low-density lipoprotein (LDL)-cholesterol (hereinafter referred to as LDL-C) was calculated using the following Equation 2. In addition, TG was analyzed using TG assay kits (Abcam, Cambridge, MA), respectively.

LDL-C=Total cholesterol−{(HDL-C)+(TG/5)} [EQUATION 2]

### 3-6. Histological Analysis

In order to identify the effect of the sample on the adipose tissue of the animal model, the epididymal white adipose tissue (eWAT) and liver fragments of the animal model were fixed in 10% formaldehyde, and then paraffin blocks were constructed and stained with hematoxylin & eosin (H&E). The size of adipocytes was expressed in average after obtaining the area of 15 adipocytes in the center of the representative image using the KFBIO Slide Manager (KFBIO, Ningbo, China).

### 3-7. Analysis of TG Content in Liver

In order to identify the effect of the sample on TG in the liver of the animal model, the hepatic tissue of the animal model was shredded, TG was extracted, and analysis was conducted using a TG assay kit (Abcam).

### 3-8. Analysis of Expression of Proteins Related to Hepatic Adipogenesis and Lipogenesis

In order to identify the effect of the sample on expression of proteins related to hepatic adipogenesis and lipogenesis in the animal model, hepatic tissues were homogenized to be used in the experiment, using a bullet blender (Next Advance, Troy, NY, USA) in a radioimmunoprecipitation assay (RIPA) buffer supplemented with 1% protease inhibitor and 1% phosphorylase inhibitor. The homogenized tissues were left at 4° C. for 50 minutes and centrifuged for 15 minutes at 4° ° C. and 15,000×g to obtain a supernatant. The same amount of protein was isolated from 10% SDS-PAGE and transferred to polyvinylidene fluoride membranes (Bio-Rad, Hercules, CA, USA). Tris-buffered saline solution with Tween 20 (hereinafter referred to as TBST, 0.1%) supplemented with 5% bovine serum albumin was blocked with a buffer solution, and then a reaction was carried out overnight at 4° C. with ACC, p-ACC, FAS, C/EBPα, PPARγ, SCD-1, SREBP-1c, DGAT, and β-actin antibodies. A reaction was carried out using horseradish peroxidase-labeled secondary antibodies at room temperature for 1 hour, followed by 4 times washing with TBST buffer. The protein band was detected by enhanced chemiluminescence detection kits (BioRad, Hercules, CA, USA), and the band intensity was corrected with β-actin protein and quantified using Image Lab software 5.1 (BioRad).

### 3-9. Analysis of Gut Microbes

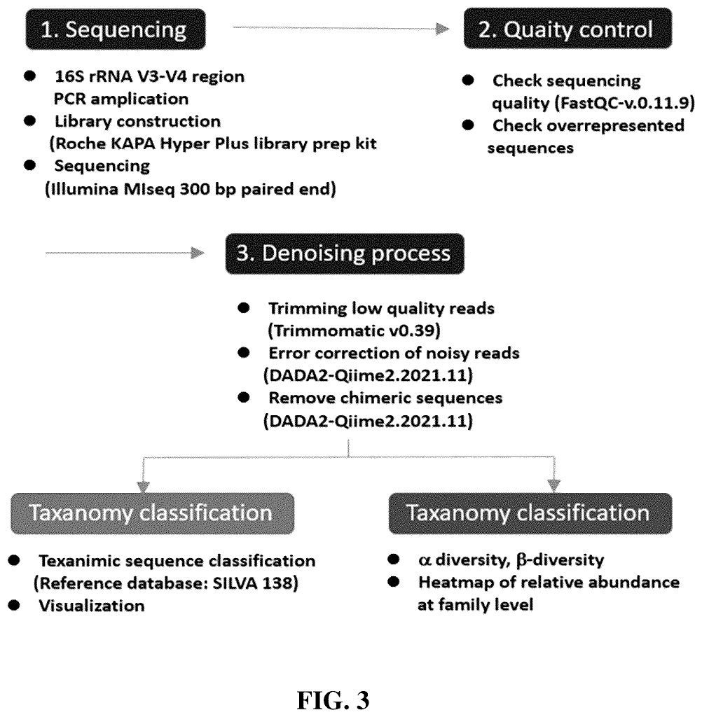

In order to identify the effect of the sample on gut microbes in the animal model, the animal model was sacrificed, the appendix was extracted, and the analysis was performed as shown in FIG. 3 using 16s rRNA metagenome sequencing.

### 3-10. Statistical Analysis

GraphPad Prism 9.4.0 (GraphPad Software Inc., San Diego, CA, USA) and SPSS statistics V. 26 (SPSS Inc., Chicago, IL, USA) were used for statistical analysis, and significant differences in treatment range (p<0.05) were analyzed using one-way analysis of variance (ANOVA) and multiple comparative tests of Duncan and Newman-Keuls.

### Example 1

### In Vitro Experiment

### 1-1. Cytotoxicity Analysis

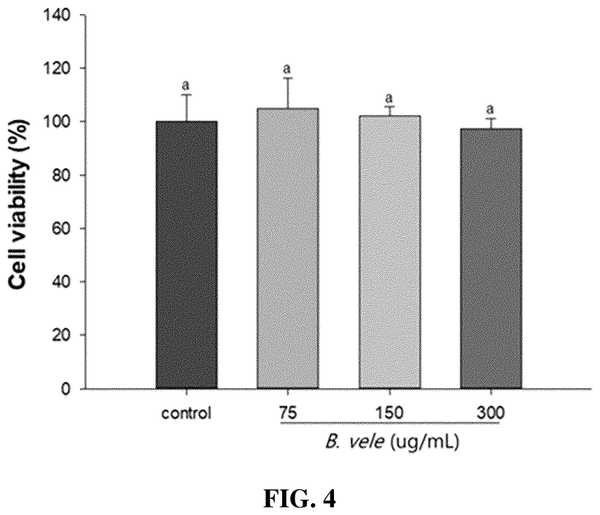

According to above Experimental Example 2-2, as a result of analyzing the cytotoxicity of the sample in adipocytes, as shown in FIG. 4, it was found that there was no significant change in cell viability up to treatment concentration of the 300 μg/mL sample (B. vele).

### 1-2. Analysis of Fat Accumulation and TG Content

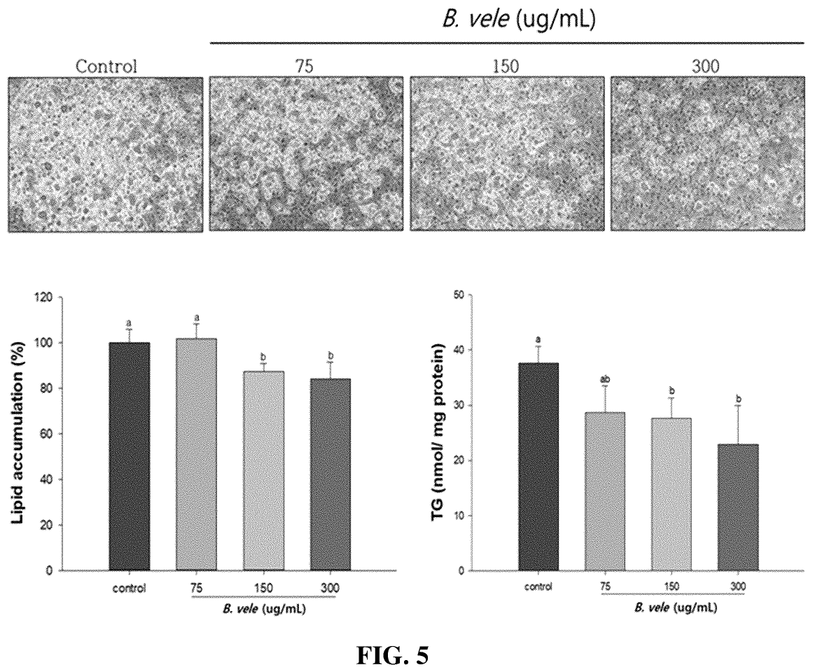

According to above Experimental Examples 2-3, as a result of analyzing the effect of the sample on accumulation of fat and TG, as shown in FIG. 5, the sample (B. vele) significantly suppressed accumulation of fat and TG in a concentration-dependent manner, and it was observed that 300 μg/mL treatment group reduced accumulation fat and TG in the cell by about 20% and 39%, respectively. In addition, from the above results, it was found that the KMU01 strain had an anti-obesity effect through suppression of fat accumulation.

### 1-3. Analysis of Expression of Adipogenesis-Related Genes and FAS

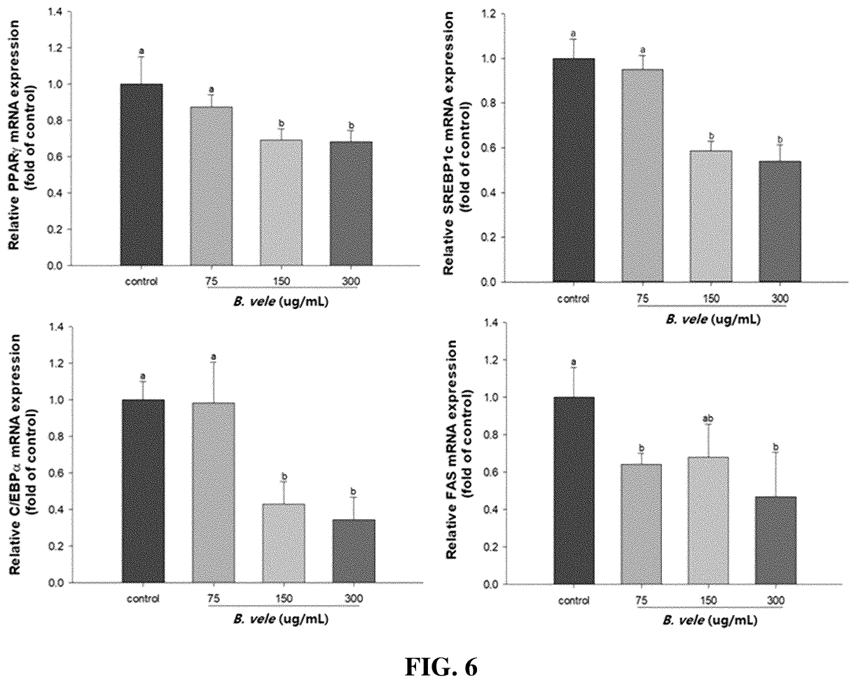

According to above Experimental Examples 2-4, as a result of analyzing the effect of the sample on expression of adipogenesis-related genes and FAS, as shown in FIG. 6, the sample (B. vele) inhibited mRNA expression of PPARγ, C/EBPα and SREBP-1c, and FAS, and specifically, the mRNA expression in the 300 μg/mL treatment group was reduced by 32%, 65%, 46%, and 53%, respectively, compared to the control group.

### Example 2

### In Vivo Experiment

### 2-1. Analysis of Weight and Dietary Efficiency

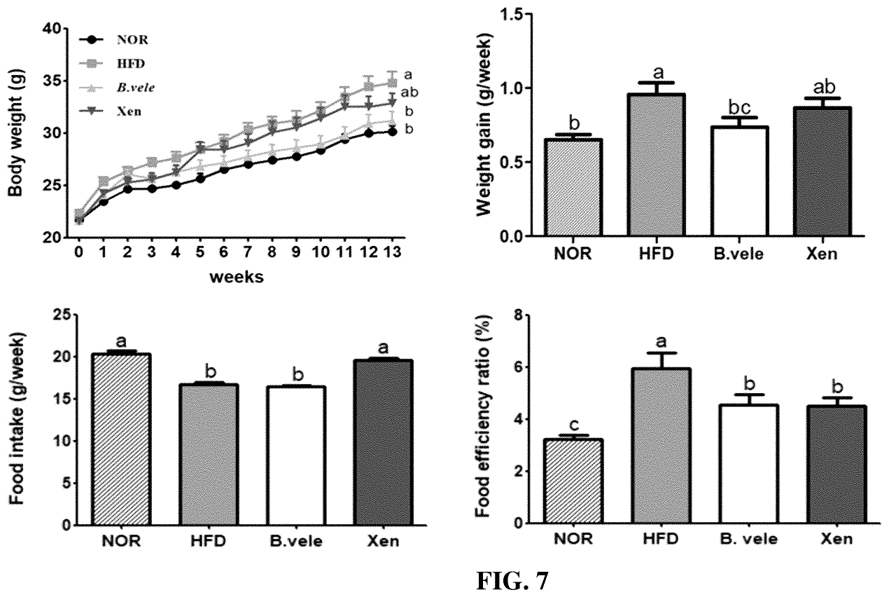

According to above Experimental Example 3-2, as a result of analyzing the effect of the sample on the weight and dietary efficiency of the animal model, as shown in FIG. 7, it was found that the body weight significantly increased in the high-fat diet group (HFD) compared to the normal diet group (NOR), indicating that obesity was induced. In addition, it was found that there was significant reduction in the body weight in the sample administration group (B. vele) compared to the high-fat diet group (p<0.05). It was found that an amount of weight gain was significantly higher in the high-fat diet group than in the normal diet group, while the sample administration group was significantly lower than the high-fat diet group (p<0.05). Food intake was not different between the high-fat diet group and the sample administration group, but the dietary efficiency was significantly low in the sample administration group (p<0.05). From the above results, it was revealed that the utilization rate of digestion and absorption rate of some nutrients was reduced by the sample, or the energy consumption increased.

### 2-2. Analysis of Body Composition

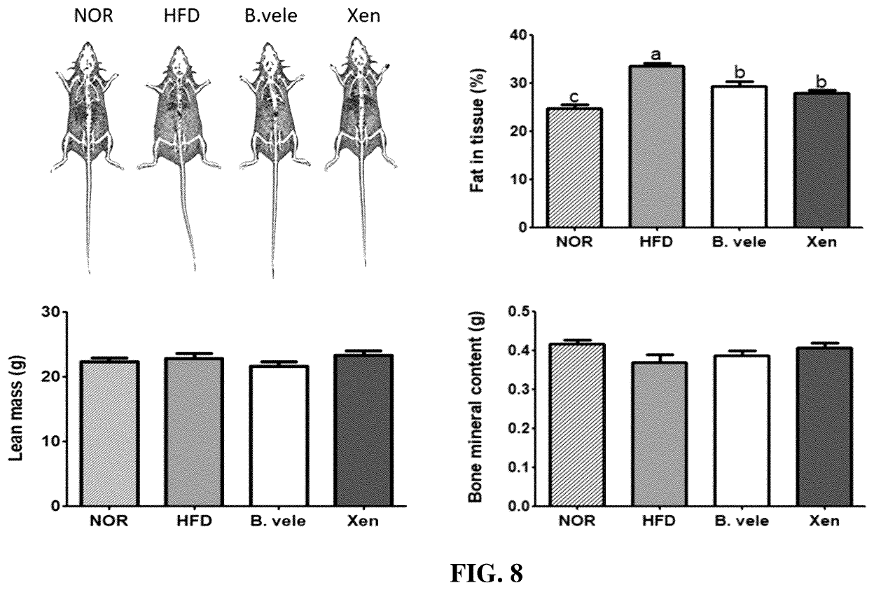

According to above Experimental Example 3-3, as a result of analyzing the effect of the sample on the body composition of the animal model, as shown in FIG. 8, body fat significantly increased in the high-fat diet group (HFD) compared to the normal diet group (NOR), and body fat was significantly reduced in the sample administration group (B. vele) and positive control group (Xen). There was no significant difference in the lean mass and bone mineral content among the experimental groups. From the above results, it was found that the weight change by the sample was due to the reduction in the body fat.

### 2-3. Analysis of Weights of Organs and Adipose Tissues

According to above Experimental Examples 3-4, as a result of analyzing the effects of the sample on organs and adipose tissues of the animal model, as shown in Table 1, the weights of the heart, liver, and spleen did not show significant differences among the experimental groups. The weight of kidney increased in the high-fat diet group (HFD), but no statistical significance was observed.

TABLE 1

| Weight (mg) | NOR | HFD | B.vele | Xen |
|---|---|---|---|---|
| Heart | 146 ± 24 | 150 ± 10 | 151 ± 23 | 157 ± 20 |
| Liver | 1104 ± 76 | 1085 ± 92 | 1047 ± 86 | 1121 ± 88 |
| Kidney | 341 ± 37 | 368 ± 46 | 355 ± 38 | 381 ± 46 |
| Spleen | 69 ± 12 | 69 ± 7 | 76 ± 13 | 73 ± 13 |

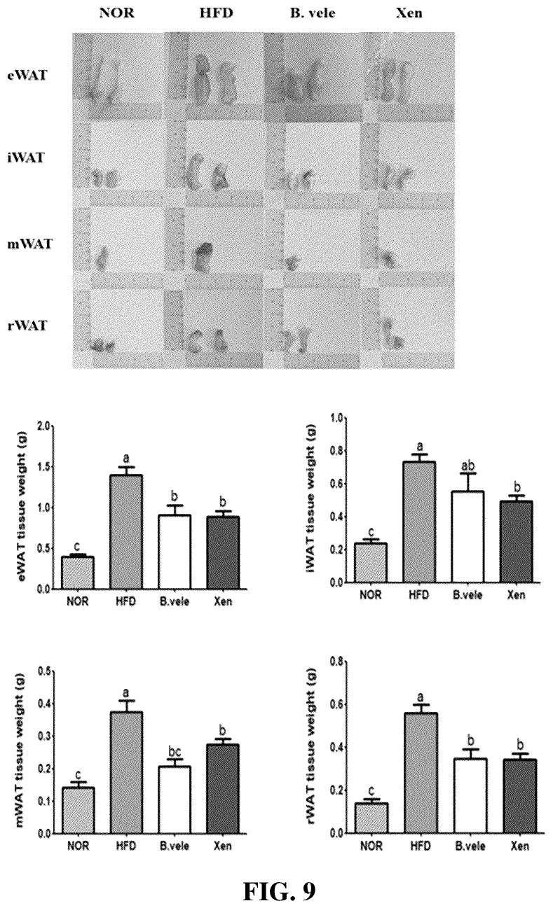

In addition, as shown in FIG. 9, the weights of all adipose tissues [(inguinal white adipose tissue (iWAT), mesenteric white adipose (mWAT), retroperitoneal white adipose tissue (rWAT), and epididymal white adipose tissue (eWAT)] of the high-fat diet group (HFD) significantly increased compared to the normal diet group (NOR), while the sample administration group (B. vele) showed decreases in the weights of all adipose tissues compared to the high-fat diet group, and there was a tendency similar to the positive control group (Xen) in which the weights of all adipose tissues decreased.

### 2-4. Biochemical Analysis of Blood

According to above Experimental Examples 3-5, as a result of analyzing the effect of the sample on blood glucose, AST, ALT and BUN, and cholesterol in the animal model, as shown in Table 2, there was no significant difference in TG in the normal diet group (NOR) and the high-fat diet group (HFD), but TG was significantly reduced in the positive control group (Xen) compared to the high-fat diet group (p<0.05). In the case of cholesterol, total cholesterol (TCHO) and HDL-C significantly increased by the high-fat diet, and a LDL-C content was significantly reduced in the sample administration group (B. vele) compared to the high-fat diet group. There were no significant differences observed in terms of indices for blood glucose, hepatosis (AST and ALT), and renal toxicity (BUN) among the experimental groups.

TABLE 2

| Indicators | NOR | HFD | B.vele | Xen |
|---|---|---|---|---|
| TG (mg/dL) | 58.41 ± 16.43 | 59.61 ± 11.76 | 50.92 ± 26.35 | 30.56 ± 8.97 |
| Total cholesterol | 150.33 ± 21.91 | 178.80 ± 9.04 | 156.40 ± 10.11 | 173.60 ± 19.24 |
| (TCHO) (mg/dL) | | | | |
| HDL-C (mg/dL) | 113.74 ± 6.09 | 131.07 ± 6.70 | 133.96 ± 2.71 | 135.94 ± 2.71 |
| LDL-C (mg/dL) | 24.91 ± 18.01 | 35.81 ± 6.36 | 12.26 ± 9.28 | 31.55 ± 17.44 |
| Blood glucose | 209.29 ± 17.40 | 217.60 ± 66.05 | 191.60 ± 29.91 | 173.56 ± 21.07 |
| (mg/dL) | | | | |
| AST (U/L) | 66.43 ± 16.69 | 69.40 ± 11.72 | 59.00 ± 15.84 | 60.22 ± 17.38 |
| ALT (U/L) | 27.71 ± 3.99 | 26.00 ± 7.18 | 20.00 ± 9.19 | 20.00 ± 2.78 |
| BUN (mg/dL) | 31.81 ± 5.30 | 26.96 ± 4.90 | 27.96 ± 6.90 | 26.78 ± 4.00 |

All values are expressed as mean ± standard deviation.

### 2-5. Histological Analysis

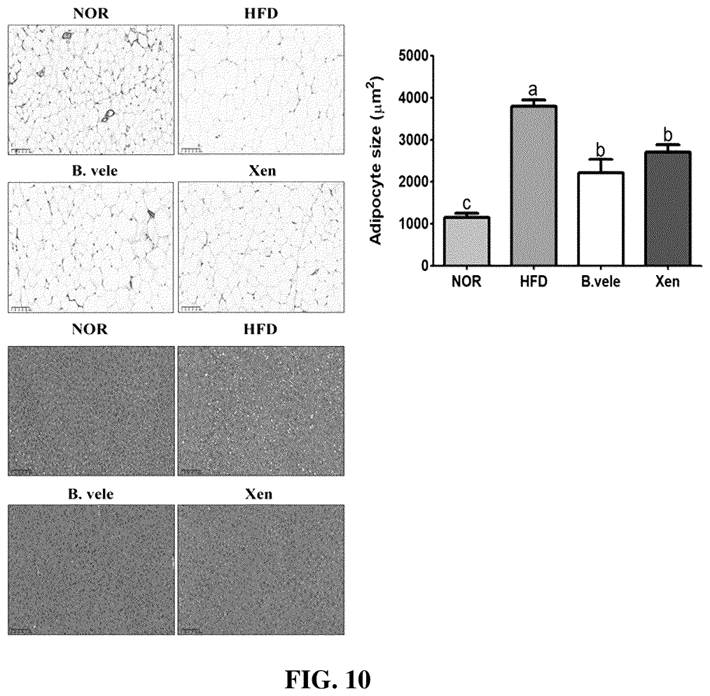

According to above Experimental Examples 3-6, as a result of analyzing the effect of the sample on adipose tissues in the animal model, as shown in FIG. 10, the size of adipocytes in the high-fat diet group (HFD) significantly increased in the epididymal white adipose (eWAT) compared to the normal diet group (NOR), and that of adipocytes was significantly reduced, compared to the high-fat diet group, in the sample administration group (B. vele) and positive control group (Xen), indicating suppression of adipocyte hypertrophy. In addition, in the liver, it was determined that the production of lipid droplets (white dots) increased in the high-fat diet group compared to the normal diet group, and the lipid droplets decreased in the sample administration group compared to the high-fat diet group.

### 2-6. Analysis of Hepatic TG Content

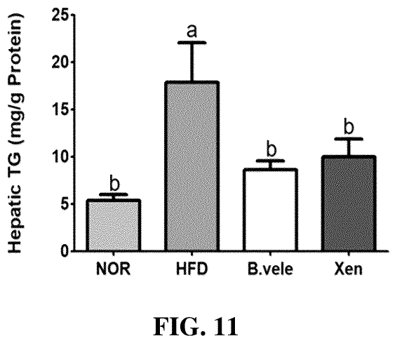

According to above Experimental Examples 3-7, as a result of analyzing the effect of the sample on hepatic TG in the animal models, as shown in FIG. 11, the TG content significantly increased in the high-fat diet group (HFD) compared to the normal diet group (NOR), and the TG content significantly decreased in the sample administration group (B. vele) and positive control group (Xen) compared to the high-fat diet group.

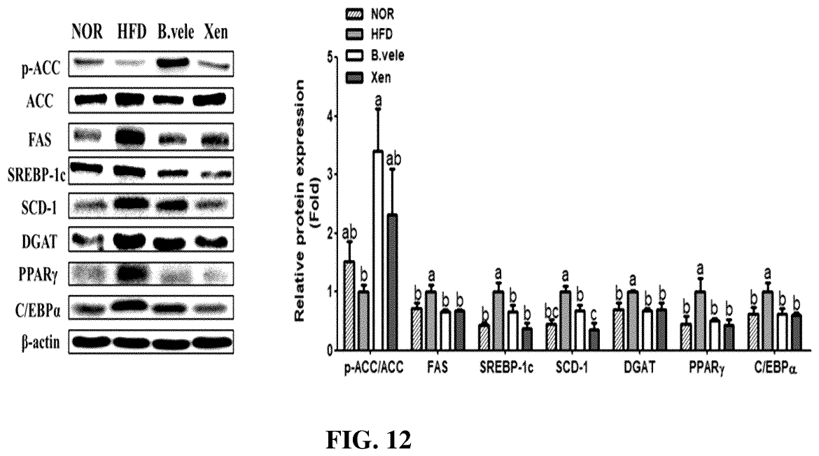

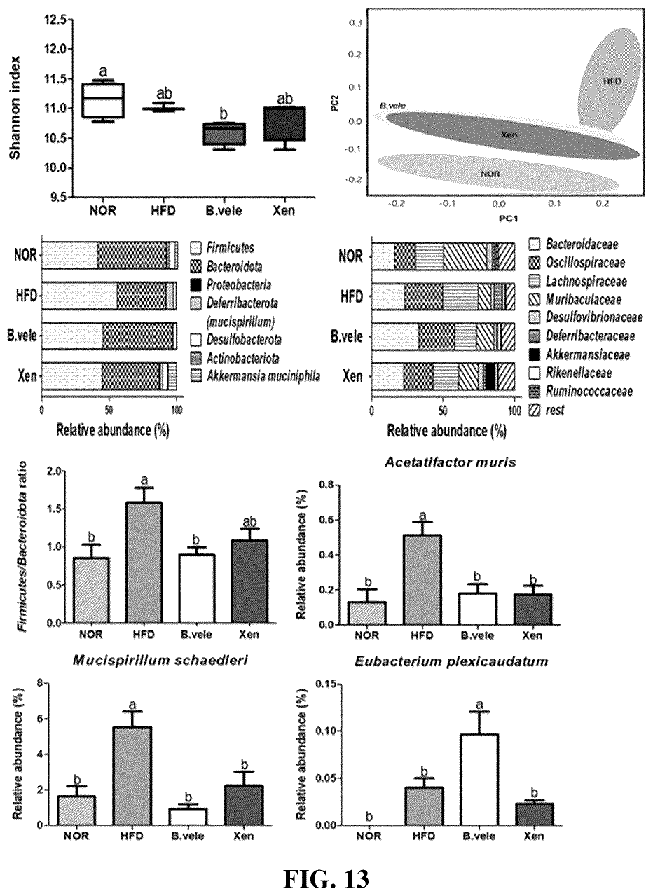

2-7. Analysis of expression of proteins related to hepatic adipogenesis and lipogenesis According to above Experimental Examples 3-8, as a result of analyzing the effect of the sample on expression of proteins related to hepatic adipogenesis and lipogenesis in the animal model, as shown in FIG. 12, it was found that the expression of adipogenesis regulatory proteins (C/EBPα and PPARγ) significantly decreased in the sample administration group (B. vele), and the expression of SREBP-1c that regulates expression of acetyl-CoA carboxylase (ACC) and fatty acid synthase (FAS) which are adipocyte synthesis enzymes (P<0.05) decreased. In addition, the expression of FAS, SCD-1, and DGAT, which are involved in the lipogenic process, was significantly reduced (P<0.05). The phosphorylation of ACC, which inhibits activation of ACC, significantly increased, revealing that the sample contributed to the inhibition of the lipogenic process. The positive control group (Xen) also showed a similar tendency as the sample administration group (B. vele).

### 2-8. Analysis of Gut Microbes

According to above Experimental Example 3-9, as a result of analyzing the effect of the sample on gut microbes in the animal model, as shown in FIG. 13A, the results of the α-diversity analysis to analyze the diversity of microbes present in one sample showed no significant difference in the Shannon index among experimental groups. On the other hand, in the β-diversity analysis results on the basis of the unweighted UniFrac principal coordinate analysis (PCA) which is frequently used for the analysis on types of microbiota, significant differences were observed between experimental groups. The administration of the sample altered the composition of the gut microbes to resemble that of the normal diet group (NOR).

In addition, the relative abundance of Deferribacterota significantly increased in the high-fat diet group (HFD) and significantly decreased in the sample administration group and the positive control group (Xen). At the phylum level, in the structural microbial analysis, the relative abundance of Firmicutes increased by high-fat diet, while Bacteroidota decreased, showing an increase in the Firmicutes/Bacteroidota (F/B) ratio and a decrease in the F/B ratio in the sample administration group. At the family level, the relative abundance of Muribaculaceae was significantly reduced in the high-fat diet group compared to the normal diet group. In the sample administration group, the relative proportion of Lachnospiraceae decreased, while that of Muribaculaceae increased. At the species level, the relative abundance of Acetatifactor muris and Mucispirillum schaedleri significantly increased in the high-fat diet group compared to the normal diet group. In the sample administration group, the relative abundance of Acetatifactor muris and Mucispirillum schaedleri was significantly reduced compared to the high-fat diet group, while that of Eubacterium plexicaudatum increased significantly.

As described above, a specific part of the content of the present disclosure is described in detail, for those of ordinary skill in the art, it is clear that the specific description is only a preferred example embodiment, and the scope of the present disclosure is not limited thereby. Thus, the substantial scope of the present disclosure may be defined by the appended claims and their equivalents.

### (Deposition Number)

Name of Depository Authority: Korea Research Institute of Bioscience and Biotechnology
Deposition Number: KCTC11751BP
Date of Deposition: Aug. 25, 2010

1. A method of preventing or treating obesity, comprising:
administering a pharmaceutical composition comprising a fermented culture supernatant of a Bacillus velezensis strain, a concentrate thereof, a dried product thereof, a fermentation metabolite thereof, or a mixture thereof as an active ingredient to a subject.

2. The method of claim 1, wherein pharmaceutical composition reduces visceral fat production.

3. The method of claim 1, wherein the pharmaceutical composition regulates one or more gut microbes selected from the group consisting of Acetatifactor muris, Mucispirillum schaedleri, and Eubacterium plexicaudatum.

4. The method of claim 2, wherein the pharmaceutical composition reduces visceral fat production by decreasing relative abundance of an Acetatifactor muris or Mucispirillum schaedleri strain in the intestine and increasing relative abundance of an Eubacterium plexicaudatum strain in the intestine.

5. The method of claim 1, wherein the strain is a Bacillus velezensis KMU01 strain deposited in deposit number KCTC11751BP.

6. The method of claim 1, wherein the pharmaceutical composition further comprises dead bacteria or spores of Bacillus velezensis.

7. The method of claim 1, wherein the fermentation metabolite is short chain fatty acids (SCFAs), organic acids, or amino acids.

8. The method of claim 7, wherein the short chain fatty acid is butyric acid or propionic acid.

9. The method of claim 7, wherein the amino acid is phenylalanine, which is an aromatic amino acid, or valine, which is a branched amino acid.

10. The method of claim 1, wherein the obesity is one or more selected from the group consisting of visceral obesity, abdominal obesity, systemic obesity, and partial obesity.

11. A method of preventing or ameliorating obesity, comprising:
administering a health functional food composition comprising a fermented culture supernatant of a Bacillus velezensis strain, a concentrate thereof, a dried product thereof, a fermentation metabolite thereof, or a mixture thereof as an active ingredient to a subject.

12. A method of preventing or ameliorating obesity, comprising:
administering a food composition comprising a fermented culture supernatant of a Bacillus velezensis strain, a concentrate thereof, a dried product thereof, a fermentation metabolite thereof, or a mixture thereof as an active ingredient to a subject.

13. (canceled)
